(19) United States
(12) Patent Application Publication
(10) Pub. No.: US 2014/0179997 A1
(43) Pub. Date: Jun. 26, 2014

(54) System with Decoupled Multiple Cameras for Use in Minimal-Invasive Surgery

(71) Applicant: avateramedical GmBH, Jena (DE)

(72) Inventors: Hubertus von Grünberg, Hannover (DE); Marcel Seeber, Jena (DE)

(73) Assignee: avateramedical GmBH, Jena (DE)

(21) Appl. No.: 13/803,252

(22) Filed: Mar. 14, 2013

(30) Foreign Application Priority Data
Dec. 20, 2012 (DE) 10 2012 025 100.9

(51) Int. Cl. A61B 1/04; A61B 1/00; A61B 1/06; A61B 19/00

(52) U.S. Cl. CPC A61B 1/042; A61B 19/2203; A61B 1/00149; A61B 1/06
USPC 600/102

(57) ABSTRACT

The present invention relates to a surgical robot system with at least two robot arms (45, 47, 49, 51), on each of which is arranged at least one endoscope for a minimally invasive surgery, wherein the first endoscope on the first robot arm (47) comprises a main support means (4b) and which comprises at the distal end at least one lighting unit (23, 24) and two image-taking devices (20a, 21a, 22a, 20b, 21b, 22b), and a trocar (1b), and wherein the second endoscope on to the second robot arm (45) comprises a main support means (4a), a trocar (1a), and an auxiliary support means (3).

### CROSS REFERENCE

The present application claims priority to German patent application serial no. DE 10 2012 025 100.9 filed Dec. 20, 2012, incorporated by reference herein in its entirety.

### FIELD OF INVENTION

The present invention relates to a multi-camera system consisting of at least one endoscope camera and at least one trocar camera for use in minimally invasive procedures, as well as an appropriate surgical robot, especially for use in minimally invasive surgery, such as laparoscopy.

### BACKGROUND

Minimally invasive surgical procedures, such as laparoscopic surgery performed with the use of surgical instruments, such as gripper tongs, cutting tools and sewing tools that are introduced via one or more trocars into the body of a patient. Usually two to four, and in most cases, three, surgical instruments are used. In addition to these surgical instruments, it is required that a display unit is present that allows the surgeon to observe the surgical field. Such a display unit is regularly a camera or an endoscope, which is also inserted through a trocar into the body of the patient. Usually, the visualization is made possible by an endoscope, which displays images of the surgical field in 2D or 3D on an external monitor. In the prior art, there exist numerous endoscopes, in which a display unit, such as a camera, is integrated in its distal end. Generally, endoscopes can have a camera at the distal as well as at its proximal end. The images obtained with the endoscope are displayed on one or more external monitors using an image transfer system and an image processing unit. Numerous endoscopes are described in the prior art.

The disadvantages of the camera systems or endoscopes described in prior art is that the endoscope is provided only for the visualization of the surgical field, but this endoscope cannot display at the same time the position and orientation of the surgical instruments inserted into the abdominal cavity, due to the varying positions the surgical instruments and the position of endoscopes in wider vicinity of the operation procedure and the field of view, whereby only the immediate area of the operation procedure is displayed. When a surgical instrument is removed from the surgical field of view, it is no longer detected by the endoscope and is no longer under the visual control of the surgeon or his assistant.

### SUMMARY OF THE INVENTION

In one aspect, the present invention provides surgical robotic systems having at least two robot arms to each of which is arranged at least one endoscope,

wherein the first endoscope on the first robot arm comprises a main support means, which extends substantially over the entire endoscope length from outside into the interior of the body, and which comprises at the distal end at least one lighting unit and two image-taking devices, wherein the image-taking devices are each pivotally mounted to the outside essentially in the same plane of the main support means, and comprise a trocar, which accomplishes the access of the first endoscope inside the body, and

wherein the second endoscope on the second robot arm comprises a main support means, which extends essentially over the entire length of the endoscope from the outside into the interior of the body, a trocar, which accomplishes the access of the second endoscope inside the body, and an auxiliary support means, which is provided on the trocar and/or the main support means, wherein the additional support means comprises at its distal end an auxiliary image-taking device, which is arranged pivotally from the additional support means to the outside, and wherein the additional image-taking device comprises an auxiliary lighting unit and at least one additional image sensor having a monitoring region, which includes the two monitoring areas of the image-taking means of the first endoscope,

wherein an image processing unit, which is connected both to the two imaging devices and the additional image-taking device, and a visualization unit is provided, which displays the 2D image data, and/or the 3D image data of the image-taking means and/or the additional image-taking means.

In one embodiment, the additional image sensor has a wide-angle lens, which in the pivoted state is arranged close to the distal end of the trocar. In another embodiment, the two imaging devices are mounted at the distal end of the main support means pivotally about a pivot axis, wherein the pivot axes are parallel to each other in one plane. In a further embodiment, the additional support means is abutting between the trocar (1a) and the main support means, in particular directly onto the main support means, wherein in particular both the main support means and the additional support means are formed cylindrical. In another embodiment, the image-taking devices are arranged by means of joints so that in each case they can be tilted both about the pivot axis and a further axis of rotation orthogonally to the longitudinal extension of the main support means, wherein the rotation about the pivot axes and the rotational axes are independent being decoupled from each other. In a still further embodiment, (i) at least one third endoscope is provided on a third robot arm, which comprises a main support means, which extends substantially over the entire endoscope length from the outside into the body, (ii) a trocar, which accomplishes the access of the third endoscope inside the body, and (iii) an additional support means, which is arranged on the trocar and/or the main support means, wherein the additional support means comprises at its distal end an additional image-taking means, which is arranged pivotably from the additional support means to the outside, and wherein the additional image-taking device comprises an additional lighting unit and at least one additional image sensor, which has a monitoring area that includes the two monitoring areas of the image-taking means of the first endoscope. In yet another embodiment, the additional imaging means of the third endoscope is connected to the image-processing unit, and the display unit displays the 2D image data and/or the 3D image data of the image-taking means and/or the additional image-taking means and/or the additional image-taking means.

### DESCRIPTION OF THE FIGURES

Purely by way of example, the present invention is now illustrated by the accompanying figures.

FIG. 1 shows a schematic view of a preferred trocar assembly during a minimally invasive procedure using a proper endoscope in a preferred embodiment of a 3D-detail camera, which is arranged on an inventive endoscope, and at least one 2D-vision camera, which is arranged on a separate support on another trocar, which are connected to an image-processing unit and a display unit of a surgical robot system.

FIG. 2 shows a schematic overview of the use of visualization solution consisting of 3D-detail camera and 2D-surveillance camera, a robotic surgical system for use in minimally invasive surgery, such as laparoscopy.

FIG. 3 shows a schematic view of a preferred trocar assembly during a minimally invasive procedure using a proper Endoscopes in a preferred embodiment of a 3D detail camera, which is arranged on an inventive endoscope, and at least two 2D-vision cameras, which are arranged on separate carriers on two different further trocar, which are connected to an image-processing unit and a display unit of a surgical robot system.

FIG. 4 shows a schematic overview of the use of the visualization solution consisting of 3D-detail camera and two 2D-surveillance cameras, a robotic surgical system for use in minimally invasive surgery, such as laparoscopy.

### DETAILED DESCRIPTION OF THE INVENTION

The technical task of the invention therefore is to provide an improved visualization system for minimally invasive surgical procedures, such as laparoscopic procedures, which provides the surgeon with the additional information about the location and orientation of instruments introduced into the patient, for example through the abdomen. This task is solved by the present invention according to claim 1 by a surgical robot system with at least two corresponding endoscopes.

The present invention provides a decoupled multi-camera system consisting of an endoscope and at least one further trocar camera for use in minimally invasive surgical procedures, such as laparoscopy.

A first subject of the present invention relates to a trocar camera for minimally invasive surgery, in particular for use within a surgical robot system with at least two robot arms, on each of which are arranged at least one endoscope for minimally invasive surgery,

wherein the first endoscope comprises in the first robot arm a main support means, which extends essentially over the entire endoscope length from the outside into the interior of the body, and which comprises at the distal end at least one lighting unit and two image-taking devices, wherein the image-taking devices are each pivotally mounted essentially in the same plane as the main support means and comprises a trocar, which accomplished the access of the first endoscope inside the body, and

wherein the second endoscope on the second robot arm comprises a main support means extending essentially over the entire endoscope length from outside into the interior of the body,

a trocar, which accomplished the access of the second endoscope inside the body, and

an additional support device, which is provided on the trocar and/or the main support means, wherein the additional support means comprises at its distal end an additional image-taking means, which is pivotally mounted on the additional support means to the outside and wherein the additional image-taking means comprises an auxiliary lighting unit and at least one auxiliary image sensor, which comprises a surveillance area that includes the two surveillance areas of the of the image-taking devices of the first endoscope,

wherein an image-processing unit, which is coupled both to the two image-taking devices and the additional of image-taking means (8, 9, 10, 11), and a visualization unit, which displays the 2D image data and/or 3D image data of the image-taking devices and/or the additional image-taking means.

The present invention has the advantage that by the provision and the simultaneous use of at least two imaging systems, at least one at least 2D vision camera at an endoscope and a 3D detail camera at another endoscope, wherein for the introduction of the 2D view camera a combination trocar is used together with a surgical instrument and are introduced into the body of a patient, it is possible to generate both an at least 2D view image with a broad field of view (wide angle of typically >90°) and a 3D detail image with a usual field of view of up to 70°. This makes it possible to monitor the direct operation area and its wider area for the entire duration of a minimally invasive surgical, such as laparoscopic, surgery. In this way, all surgical instruments can be imaged simultaneously, even if—due to their varying positions and the position of the endoscope and the field of view—they are located outside the operating field of view of the endoscope, because the additional image-taking device can detect also instruments, which are located outside of the surgical field of view of the endoscope. This may be the case, for example, when a surgical instrument is temporarily “parked”, because it is not needed. This “parking” is in most cases out of the direct operation procedure and outside of the operation field of view so that it is not in the way during the procedure. According to the invention, such “parked” surgical elements are captured by the novel 2D surveillance camera and are thus continuously under visual control of the surgeon or his assistant.

Furthermore, the additional illumination unit on the additional support means achieves improved illumination in particular for the 3-D images so that the images of the 3D-detail camera can be represented qualitatively improved.

According to a preferred embodiment of the invention, the additional image sensor has a wide-angle lens, which in the pivoted state is arranged close to the distal end of the trocar.

A third subject of the present invention relates to a surgical robot system with at least two robot arms, on which a surgical instrument and an endoscope for minimally invasive surgery can be disposed, wherein in addition, on the trocar for the surgical instrument an additional support means is arranged with the additional image-taking device, a trocar, which accomplishes the access of the endoscope inside the body, and wherein the additional support means comprises at its distal end an additional image-taking device, which is mounted pivotally from the auxiliary support means to the outside, and wherein the additional image-taking means comprises an auxiliary lighting unit and at least one additional image sensor, which has a surveillance area that includes all surgical instruments and/or endoscopes in their positions and orientation that were introduced, for example, through the abdomen,

wherein an image-processing unit, which is coupled both to the endoscope and the additional image-taking device, and a display unit is provided, which represents 2D image data, and/or 3D image data of the endoscope and/or the additional of image-taking means.

A control unit knows the current position and orientation of the robot arms and the attached instruments or endoscopes, and this information is sent to an image-processing unit, which is coupled both to the endoscope and the additional image-taking device, and a display unit is provided that displays the 2D image data and/ or 3D image data of the endoscope and/or the additional image-taking means and, in addition, calculate from the position and orientation of the robot arms and the attached instruments or endoscope the movement trajectories and displays them as an overlay display along with the 2D image data and/or 3D image data.

The inventive surgical robot system has the particular advantage that the image data can be displayed to the surgeon as required, that is either as a 2D image data or as 3D image data, which means that the image data of the surveillance camera can also be coupled with the image data of the 3D-detail camera using the image-processing unit so as to provide the surgeon with a much improved overview by a single sequence of images on the display unit. The surgical robot system can be used, for example, in conducting minimally invasive surgery.

It is particularly advantageous when the additional image sensor has a wide-angle lens, which in the swung-out state is arranged close to the distal end of the trocar.

It is particularly advantageous if the two image-taking devices are each arranged at the distal end of the main support means pivotally mounted about a rotational axis, wherein the pivot axes are parallel to each other in one plane, whereby the construction cost is minimized.

A further structural simplification can be seen in the fact that the additional support means is provided between the trocar and the main support means, in particular directly abutting the main support means, wherein in particular both the main support means and the additional support means are cylindrical.

Furthermore, it is advantageous if the image-taking devices are tiltably arranged, by means of hinges, in each case both to the pivot axis as well as a further rotation axis orthogonal to the longitudinal extension of the support means, wherein the rotational movements about the pivot axes and the axes of rotation are independently decoupled from each other.

A further preferred embodiment of the invention is characterized in that at least a third endoscope is provided on a third robotic arm, which comprises a main support means, which extends essentially over the entire endoscope length from outside into the interior of the body, a trocar, which allows access of a third endoscopes inside the body, and an additional support means, which is provided on the trocar and/or the main support means, wherein the additional support means comprises at its distal end an additional image-taking means, which is arranged pivotally from the additional support means to the outside, and wherein the additional image-taking means comprises an additional illumination unit and at least one additional image sensor, which captures a monitoring area that includes the two control areas of the image-taking devices of the first endoscope.

Further, it is advantageous if the additional image-taking device of the third Endoscopes coupled to the image processing unit, and the display unit is the 2D image data, and/or 3D image data of the image-taking devices and/or the addition of image-taking device and/or the addition of image-taking means.

The entire disclosure of the present invention therefore relates equally to the combination consisting of a 3D-detail camera and at least one further 2D-vision camera, which is introduced into the interior of the body preferably through a further trocar used for a surgical instrument and is positioned in such a way that all further surgical instruments or endoscope cameras introduced into the body through other trocars are covered by the 2D surveillance camera optically, as well as on the combination of a 3D detail camera with at least two 2D vision cameras which preferably has two further, for surgical instruments used Trocar e, are introduced into the interior of the body and are positioned in such a way that all imported via further trocar e in the body surgical Instruments and Endoscope cameras are optically covered by the 2D surveillance camera.

In minimally invasive surgical procedures, such as laparoscopic surgery, access into the body of a patient (usually through the abdomen or the chest cavity) is created through a trocar. Using such a trocar, a surgical instrument or a camera or an endoscope can be introduced into the body. As mentioned, according to the invention a surgical instrument and a trocar camera are simultaneously introduced through a trocar. Since, as a rule, 3 to 5 surgical instruments and at least one camera are required for the surgical intervention, 3 to 5 trocars are needed.

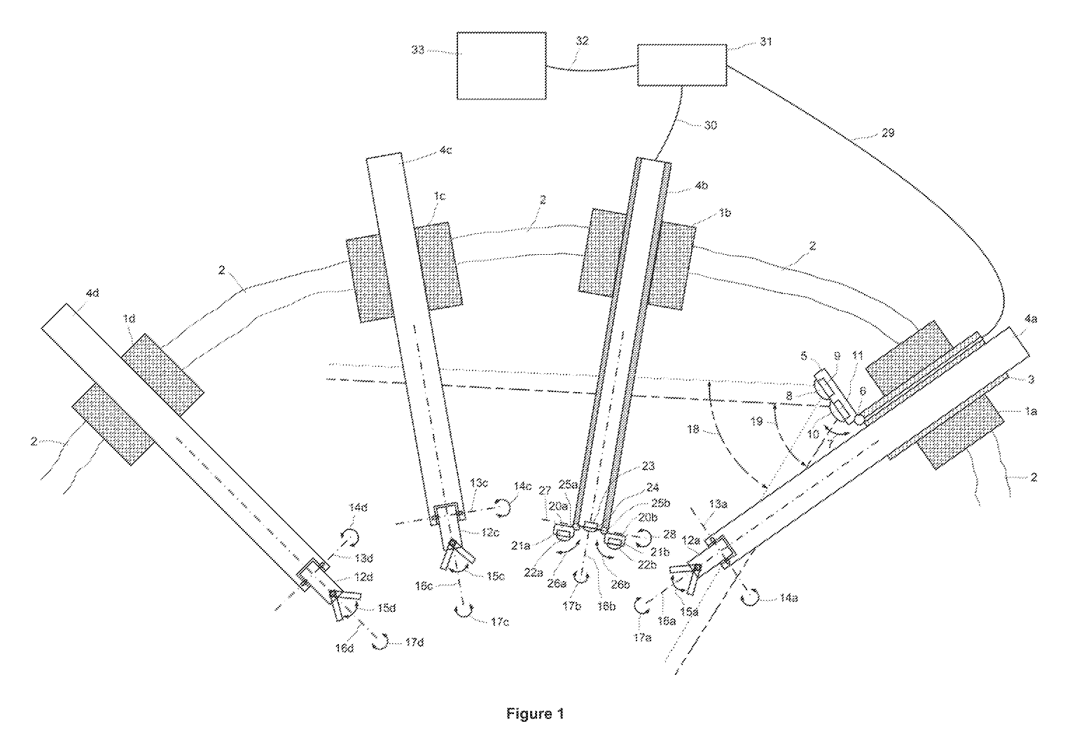

FIG. 1 shows the inventive multi-camera system. Using a trocar 1, a passage is made through the body tissue 2 and thus access to the interior of the body of a patient is provided. Through the trocar 1a, an additional support 3 for a 2D-surveillance camera is introduced into the body. The additional support 3 is designed so that it enables a tubular passage for a rotationally symmetrical rod-shaped further main support 4a for a surgical instrument. On the additional support 3 is mounted a camera holder 5 by means of a hinge 6 so that, after passage through the trocar 1a, it can be expanded essentially at 90° to the rotation axis by a pivoting movement 7. The camera holder 5 carries an additional image sensor consisting of an image sensor 9 and wide-angle imaging optics 8 with the opening angle 18. In order to illuminate the field of view, the camera holder 5 is further provided with an additional illumination unit 11 consisting of a light source 11 and a corresponding wide-angle imaging optical system 10 with the aperture angle 19. This wide-angle imaging optical system 10 is designed so that, except for the parallax offset between the wide-angle imaging lens 8 and the wide-angle imaging optical system 10, the whole field of view covered by the image sensor 9 and the associated wide angle imaging lens 8 is illuminated. The camera holder 5 with the additional image sensor and the additional lighting unit together form the 2D-surveillance camera for generating a 2D-overview image. Preferably, the image sensor 9 is designed as a CCD or CMOS sensor with a resolution of 1920×1080 pixels or higher. With an appropriate positioning of the 2D-surveillance camera at an outer trocar 1a, any further imported surgical instruments or endoscopes introduced into the body through the trocars 1b, 1c, 1d are in the field of view of the 2D-surveillance camera and can be visually captured by them and displayed on the image sensor 9.

The received image data is supplied via the data link 29 to a processing unit 31, which processes the image data for display and supplies it via a further data path 32 to a visualization unit 33. The visualization unit 33 can display both 2D and 3D image data, for example separately, but also combined into a single image or a single image sequence.

At the end of the rotationally symmetrical main support 4b are located two camera modules and two image-taking devices 20a, 21a, 22a, 20b, 21b, 22b that each consist in particular of two optical imaging systems 22a and 22b that are mounted on two camera holders 20a and 20b. The camera holders 20a and 20b are so connected to the main support 4b by means of the joints 25a and 25b, which form the pivot axes, that after the introduction into the body, they can be folded out by 90° to the rotation axis of the main support 4b in the pivoting directions 26a and 26b. In order to illuminate the field of view, at the end of the main support 4b, to which are also attached the folding camera holders 20a and 20b, there is mounted a lighting unit consisting of the light source 23 and an imaging optical system 24. The camera holders 20a and 20b continue to wear the imagers consisting of image sensors 21a and 21b and imaging optics 22a and 22b. Together, these two image-taking devices 20a, 21a, 22a, 20b, 21b, 22b form the 3D-detail camera.

The lighting unit consisting of a light source 23 and imaging optics 24 can be preferably formed as a direct LED light source in such a way that the emission angle of the LED in conjunction with a suitable imaging lens 24 is so selected that the field of view displayed by two image sensors 21a and 21b and the associated imaging optics 22a and 22b is completely illuminated.

The received image data is supplied via the data link 30 to a processing unit 31, which processes the image data for display and supplies it via a further data path 32 to a visualization unit 33. The visualization unit 33 can display both 2D and 3D image data, for example separately, but also combined into a single image or a single image sequence.

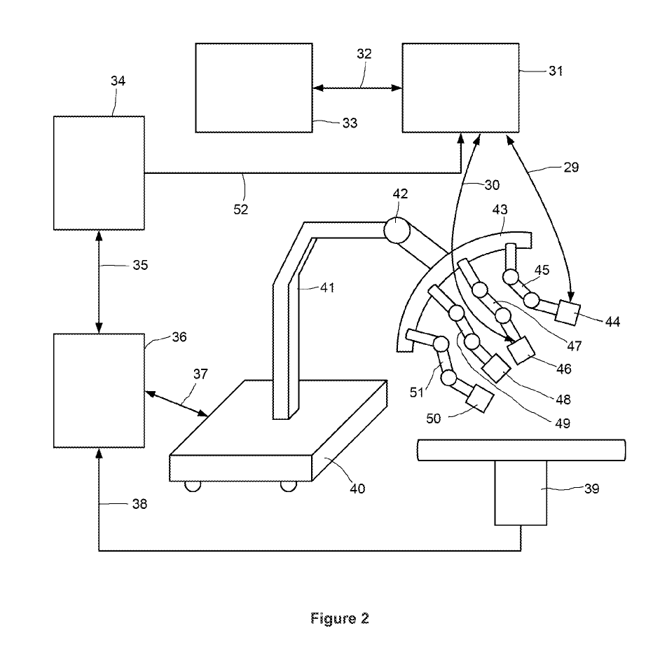

FIG. 2 shows the use of the visualization solution consisting of 3D-detail camera and a 2D surveillance camera in a surgical robotic system for use in minimally invasive surgery, such as laparoscopy. Shown is an embodiment of the robot system with 4 robotic arms 45, 47, 49, 51 and 4 trocar accesses 44, 46, 48, 50, wherein 44 comprises the 2D surveillance camera shown in FIG. 1 at the trocar 1a, 46 comprises 3D detail camera shown in FIG. 1 at trocar 1b, 48 and 50 comprises the trocars 1c, 1d for access by two other surgical instruments 4c, 4d. The access 44 for the 2D surveillance camera is connected by a pre-positioning device 45 to the guide 43. Access 46 for the 3D-detail camera is connected by means of a pre-positioning device 47 to the guide 43. The access 48 for a surgical instrument is connected to the guide 49 by means of a pre-positioning device 43. The access 50 for a surgical instrument is connected to the guide 43 by means of a pre-positioning device 51. The pre-positioning device can be realized passively, i.e., by manual adjustment, or actively. The pre-positioning device itself is held by means of a suitable support, e.g., by the guide 43. This guide 43 can be positioned by means of the joint 42 to the patient. The boom 41 is connected to the mobile support system 40, thus enabling positioning of the entire support system relative to the operating table 39. Using the control and display unit 34, the operator is advised of the current status of the pre-positioning device. Using the control and display unit 34, the operator can enter control commands, which are the sent through a suitable data link 35 to the control unit 36 and from it to the access for the 2D-vision camera 44, to the access for the 3D-detail camera 46, to the accesses 48, 50, to the pre-positioning device 45, 47, 49, 51 and to the guide 43 for further processing. The control unit 36 is connected by means of a suitable data link 37 to the support system. The operating table 39 can be also connected for control purposes by means of the data link 38 to the control unit 36 in order to be able, in case of a change in the operating table position, for example its height, to process this position change in the control unit and to signal it. Therefore, changes in the patient's position can be evaluated on the basis of a change in the position of the operating table 39.

The received image data is supplied through the data lines 29, 30 to a processing unit 31, which processes the image data for display, and supplies it via a further data path 32 to a visualization unit 33. The visualization unit 33 can display both 2D and 3D image data, for example separately, but also combined into a single image or a single image sequence.

The control and display unit 34 is connected by a suitable data link 52 to the processing unit 31. Using the control and display unit 34, the surgeon can send control commands for selection, processing and display of the image data to the processing unit 31. The processing unit 31 is connected by a suitable data link 32 to the display unit 33. The display unit 33 can display detail images/image sequences supplied by the 2D surveillance camera and the 3D camera, as well as additional information generated in the processing unit 31, such as trajectories of the surgical instruments, either as separate images or image sequence or as image information calculated by the 2D surveillance camera and/or the 3D-detail camera.

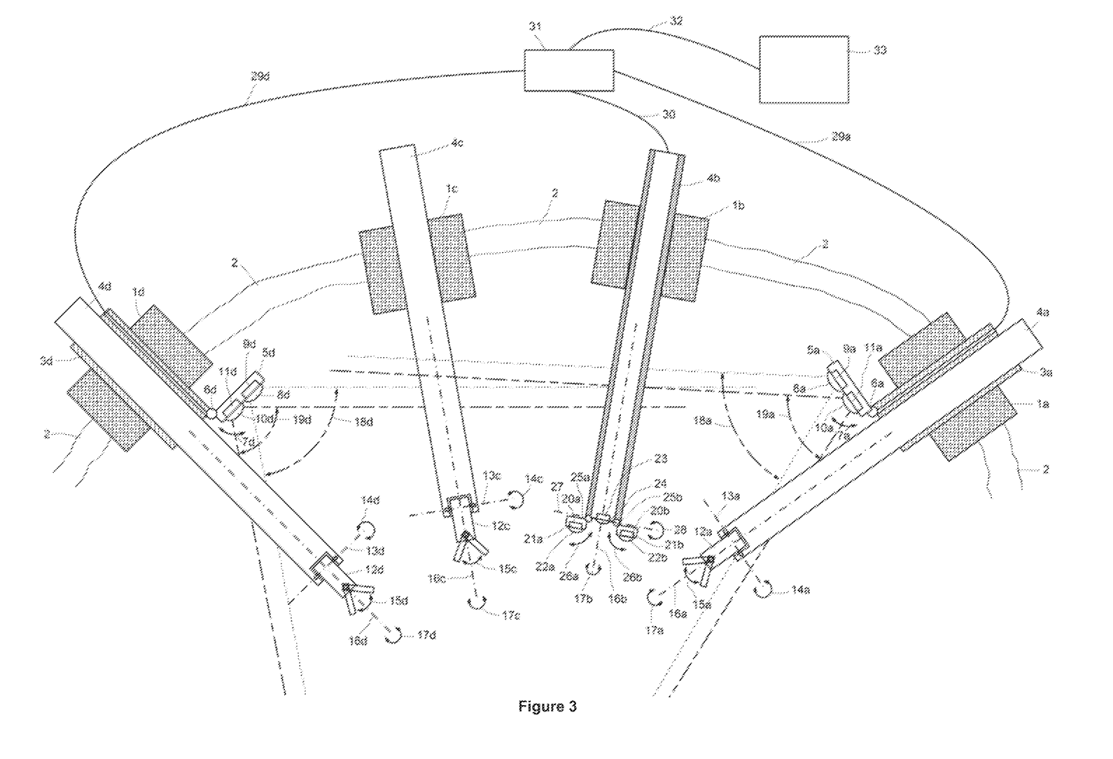

FIG. 3 shows the inventive multi-camera system. A trocar 1 provides a passage through the body tissue 2 and thus access to the interior of the body of a patient. Through the trocar 1a, an additional support 3a for a first 2D surveillance camera is introduced into the body. The additional support 3a is designed such that it allows a tubular passage for a rotationally symmetrical rod-shaped further main support 4a for a surgical instrument. To the additional support 3a is attached a camera holder 5a by a joint 6 so that, after the introduction through the trocar 1a, it can be folded out essentially at 90° to the axis of rotation by a pivoting motion 7a. The camera holder 5a carries an additional image sensor that consists of an image sensor 9a and wide-angle imaging optics 8a with the opening angle 18a. In order to illuminate the field of view, the camera holder 5a is further provided with an additional lighting unit that consists of a light source 11a and a corresponding wide-angle imaging lens 10a with the opening angle 19a. This wide-angle imaging lens 10a is configured such that, with the exception of the parallax offset between the wide-angle imaging lens 8a and the wide-angle imaging optical system 10a, the whole field of view of the image sensor 9 and the associated wide-angle imaging lens 8a is illuminated. The camera holder 5a with the additional image sensor and the additional lighting unit together form the first 2D-view camera for generating a first 2D overview image. Preferably, the image sensor 9a is designed as a CCD or CMOS sensor with a resolution of 1920×1080 pixels or higher. With a suitable positioning of the first 2D surveillance camera at an outer trocar 1a, all further surgical instruments or endoscopes introduced into body through the trocars 1b, 1c, 1d can be displayed in the field of view of the first 2D surveillance camera and can visually captured by it and imaged in the image sensor 9.

The received image data is supplied through the data line 29a to a processing unit 31, which processes the image data for display and supplies it via a further data path 32 to a visualization unit 33. The visualization unit 33 can display both 2D and 3D image data, for example separately, but also combined into a single image or a single image sequence.

A trocar 1d provides a passage through the body tissue 2 and, therefore, access to the interior of the body of a patient. An additional support 3d for a second 2D vision camera is introduced into the body through the trocar 1d. The additional support 3d is designed so that it enables a tubular passage for a further rotationally symmetrical rod-shaped main support 4d for a surgical instrument. To the auxiliary support 3d is attached a camera holder 5d by means of a hinge 6d so that after passage through the trocar 1d, it can be folded out in a pivoting movement essentially 90° to the rotation axis. The camera holder 5d carries an additional image sensor consisting of image sensor 9d and wide-angle imaging optics 8d with the opening angle 8d. In order to illuminate the field of view, the camera holder 5d is further provided with an additional lighting unit 11d consisting of a light source 11d and a corresponding wide-angle imaging optical system 10d with the opening angle 19d. This wide-angle imaging optical system 10d is designed such that, except for the parallax offset between the wide-angle imaging lens 8d and the wide-angle imaging optical system 10d, the whole field of view of the image sensor 9d and the associated wide angle imaging optics 8d is illuminated. The camera holder 5d with the additional image sensor and the additional lighting unit together form the second 2D view camera for generating a second 2D overview image. Preferably, the image sensor 9d is designed as a CCD or CMOS sensor with a resolution of 1920×1080 pixels or higher. With a suitable positioning of the second 2D surveillance camera at an outer trocar 1d, all further surgical instruments or endoscopes introduced into the body through the trocars 1a, 1b, 1c are in the field of view of the second 2D surveillance camera and can be visually captured by it and imaged onto the image sensor 9d.

The received image data is supplied by the data link 31 to a processing unit 29d, which processes the image data for display, and supplies it via a further data path 32 to a visualization unit 33. The visualization unit 33 can display both 2D and 3D image data, for example separately, but also combined into a single image or a single image sequence.

At the end of the rotationally symmetrical main support 4b are located two camera modules or two image-taking devices 20a, 21a, 22a, 20b, 21b, 22b that each consist in particular of two optical imaging systems 22a and 22b that are mounted on two camera holders 20a and 20b. The camera holders 20a and 20b are connected—by means of the joints 25a and 25b, which form the pivot axes—to the main support 4b that, after the introduction into the body, it can be folded out by 90° to the rotation axis of the main support in the pivoting directions 26a and 26b. In order to illuminate the field of view, at the end of the main support 4b, to which are also attached the folding camera holders 20a and 20b, there is provided a lighting unit consisting of the light source 23 and an imaging optical system 24. The camera holders 20a and 20b further carry imager consisting of image sensors 21a and 21b and imaging optics 22a and 22b. Together, these two image-taking devices 20a, 21a, 22a, 20b, 21b, 22b form the 3D-detail camera.

The lighting unit consisting of a light source 23 and the imaging optics 24 can preferably be realized as a direct LED light source in such a way that the emission angle of the LED in conjunction with a suitable imaging lens 24 can be selected so that the field of view formed by the two image sensors and 21a and 21b and the associated imaging optics 22a and 22b is completely illuminated.

The received image data is supplied by the data link 30 to a processing unit 31, which processes the image data for display and supplies it via a further data path 32 to a visualization unit 33. The visualization unit 33 can display both 2D and 3D image data, for example separately, but also combined into a single image or a single image sequence.

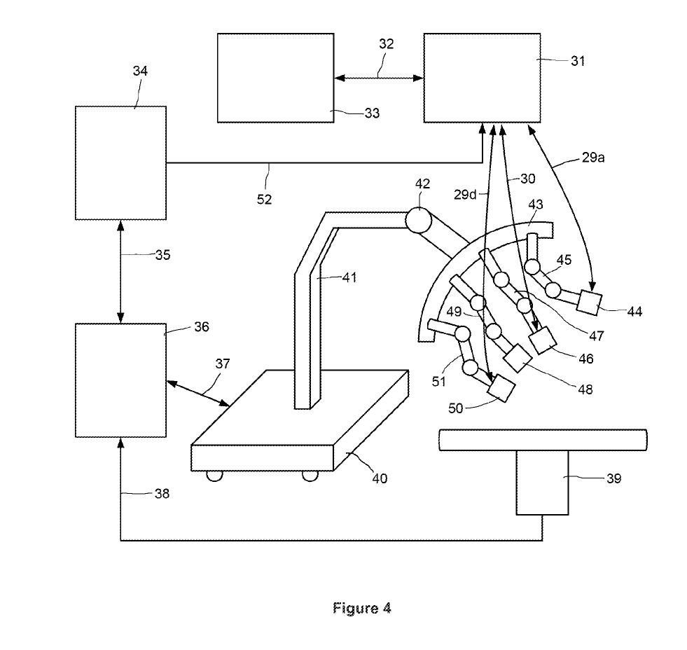

FIG. 4 shows the use of the visualization solution consisting of a 3D-detail camera and two 2D surveillance cameras in a robotic surgical system for use in minimally invasive surgery, such as laparoscopy. Shown is an embodiment of the robot system with 4 robotic arms 45, 47, 49, 51 and 4 trocar-accesses 44, 46, 48, 50, 44, wherein 44 comprises first 2D surveillance camera at the trocar 1a shown in FIG. 3, 46 comprises the 3D-detail camera at the trocar 1b shown in FIG. 1, 50 comprises the second 2D surveillance camera at the trocar 1d shown in FIG. 3, and 48 comprises trocar 1c for access of another surgical instrument 4c. The access 44 of the first 2D-view camera is connected by means of a guide 45 to the pre-positioning device 43. Access 46 for the 3D camera is connected by means of a pre-positioning device 47 to the guide 43. The access 48 for a surgical instrument is connected to a guide 49 by means of the pre-positioning device 43. Access 50 for the second 2D surveillance camera is connected by a pre-positioning device 51 to the guide 43. The pre-positioning device can be realized passively, i.e., by manual adjustment, or actively. The pre-positioning device itself is held by means of a suitable support, e.g., as a guide 43. This guide 43 can be positioned to the patient by means of the joint 42. The boom 41 is connected to the mobile support system 40, thus enabling positioning of the entire support system relative to the operating table 39. The control and display unit 34 advises the operator of the current status of the pre-positioning device. Using the control and display unit 34, the operator can enter control commands, which are sent over a suitable data link 35 to the control unit 36 and from it to the access for the 2D vision cameras 44 and 50, from it to the access for the 3D-detail camera 46, to the access 48, to the pre-positioning directions 45, 47, 49, 51 and to the guide 43 for further processing. The control unit 36 is connected to the support system by means of a suitable data link 37. The operating table 39 can be connected for control purposes via the data link 38 also to the control unit 36 so in case of a change in the operating table position, for example the height, this position change can be processed and signaled in the control unit. This allows to evaluate changes in the patient's position on the basis of a change in position of the operating table 39.

The received image data is fed through the data lines 29a, 29b, 30 to a processing unit 31, which processes the image data for display and supplies it via a further data path 32 to a visualization unit 33. The visualization unit 33 can display both 2D and 3D image data, for example separately, but also combined into a single image or a single image sequence.

The control and display unit 34 is connected by a suitable data link 52 to the processing unit 31. Using the control and display unit 34, the surgeon can send control commands for selection, processing and display of the image data to the processing unit 31. The processing unit 31 is connected by a suitable data link 32 to the display unit 33. The display unit 33 can be supplied by the 2D surveillance camera and the 3D-detail camera with images or image sequences as well as additional information generated in the processing unit 31, such as the trajectories of the surgical instruments that are represent either as separate images/image sequence or as images or image sequences calculated by the image information of the 2D surveillance camera and/or the 3D-camera detail.

Thus, the present invention describes a surgical robot system, in which the trajectories of the surgical instruments and the lighting devices are displayed on a display of the display unit so that the surgeon is informed not only of the current position of the individual elements of the instruments but also gets displayed, in which direction are other instruments and lighting equipment. Thus, the present invention makes it possible for the surgeon to always coordinate all instruments and not to have to bring them to the field of view of the 3D camera in a “blind flight”.

1. A surgical robotic system having at least two robot arms to each of which is arranged at least one endoscope,
wherein the first endoscope on the first robot arm comprises a main support means, which extends substantially over the entire endoscope length from outside into the interior of the body, and which comprises at the distal end at least one lighting unit and two image-taking devices, wherein the image-taking devices are each pivotally mounted to the outside essentially in the same plane of the main support means, and comprise a trocar, which accomplishes the access of the first endoscope inside the body, and
wherein the second endoscope on the second robot arm comprises a main support means, which extends essentially over the entire length of the endoscope from the outside into the interior of the body, a trocar, which accomplishes the access of the second endoscope inside the body, and an auxiliary support means, which is provided on the trocar and/or the main support means, wherein the additional support means comprises at its distal end an auxiliary image-taking device, which is arranged pivotally from the additional support means to the outside, and wherein the additional image-taking device comprises an auxiliary lighting unit and at least one additional image sensor having a monitoring region, which includes the two monitoring areas of the image-taking means of the first endoscope,
wherein an image processing unit, which is connected both to the two imaging devices and the additional image-taking device, and a visualization unit is provided, which displays the 2D image data, and/or the 3D image data of the image-taking means and/or the additional image-taking means.

2. The robot system according to claim 1, characterized in that the additional image sensor has a wide-angle lens, which in the pivoted state is arranged close to the distal end of the trocar.

3. The robot system according to claim 1, characterized in that the two imaging devices are mounted at the distal end of the main support means pivotally about a pivot axis, wherein the pivot axes are parallel to each other in one plane.

4. The robot system according to claim 1, characterized in that the additional support means is abutting between the trocar (1a) and the main support means, in particular directly onto the main support means, wherein in particular both the main support means and the additional support means are formed cylindrical.

5. The robot system according to claim 1, characterized in that the image-taking devices are arranged by means of joints so that in each case they can be tilted both about the pivot axis and a further axis of rotation orthogonally to the longitudinal extension of the main support means, wherein the rotation about the pivot axes and the rotational axes are independent being decoupled from each other.

6. The robot system according to claim 1, characterized in that at least one third endoscope is provided on a third robot arm, which comprises
a main support means, which extends substantially over the entire endoscope length from the outside into the body,
a trocar, which accomplishes the access of the third endoscope inside the body, and an additional support means, which is arranged on the trocar and/or the main support means, wherein the additional support means comprises at its distal end an additional image-taking means, which is arranged pivotably from the additional support means to the outside, and wherein the additional image-taking device comprises an additional lighting unit and at least one additional image sensor, which has a monitoring area that includes the two monitoring areas of the image-taking means of the first endoscope.

7. The robot system according to claim 6, characterized in that the additional imaging means of the third endoscope is connected to the image-processing unit, and the display unit displays the 2D image data and/or the 3D image data of the image-taking means and/or the additional image-taking means and/or the additional image-taking means.
